(19) United States
(12) Patent Application Publication
(10) Pub. No.: US 2025/0250521 A1
(43) Pub. Date: Aug. 7, 2025

(54) MICROPHYSIOLOGICAL ORGANOID MODEL

(71) Applicant: The Johns Hopkins University, Baltimore, MD (US)

(72) Inventors: Mark J. Ranek, Columbia, MD (US); Marcus Alonso Cee Williams, Baltimore, MD (US); Vivek Jani, Baltimore, MD (US); Brian Lin, Baltimore, MD (US)

(21) Appl. No.: 18/855,141

(22) PCT Filed: Apr. 10, 2023
(86) PCT No.: PCT/US23/18088

§ 371 Date: Oct. 8, 2024

Related U.S. Application Data
(60) Provisional application No. 63/329,251, filed on Apr. 8, 2022.

(51) Int. Cl. C12M 3/00; C12M 3/06; G01N 33/50

(52) U.S. Cl. CPC C12M 21/08; C12M 23/16; G01N 33/5082

(57) ABSTRACT

The disclosed in vitro systems allow for multiple organs (organoids) to be cultured in the same conditions, connected with the same microcirculation, subjected to the same pathological state, and treated with the same therapeutic approach. This allows for determination of the how various organ systems respond to a pathological stress and also how a therapeutic approach may effect various organoids differently. The in vitro systems also provide for single organoids.

### CROSS-REFERENCE TO RELATED APPLICATION

This application claims the benefit of U.S. provisional application No. 63/329,251 filed Apr. 8, 2022, which is incorporated by reference herein in its entirety.

### FIELD

Methods of tissue engineering, and more particularly methods and compositions for generating various 3D tissues, such as 3D organoids are described.

### BACKGROUND

The ability to create three-dimensional (3D) vascularized tissues on demand could enable scientific and technological advances in tissue engineering, drug screening, toxicology, 3D tissue culture, and organ repair. To produce 3D engineered tissue constructs that mimic natural tissues and, ultimately, organs, several key components—cells, extracellular matrix (ECM), and vasculature—may need to be assembled in complex arrangements. Each of these components plays a vital role: cells are the basic unit of all living systems, ECM provides structural support, and vascular networks provide efficient nutrient and waste transport, temperature regulation, delivery of factors, and long-range signaling routes.

Even after complete dissociation, cells can reaggregate and reconstruct the original architecture of an organ. More recently, this outstanding feature was used to rebuild organ parts or even complete organs from tissue or embryonic stem cells. Such stem cell-derived three-dimensional cultures are called organoids. Because organoids can be grown from human stem cells and from patient-derived induced pluripotent stem cells, they have the potential to model human development and disease and in a tree-dimensional, biomimetic environment (Lancaster M A, et al., Cerebral organoids model human brain development and microcephaly. Nature 501 (7467): 373-9 (2013)). Furthermore, they have potential for drug testing and even future organ replacement strategies (Lancaster et al., 2013). The organoids are often developed in spinning bioreactors.

### SUMMARY

Embodiments are directed to methods and compositions for producing tissues and organoids that simulate or mimic mammalian organs and their functions. In certain embodiments, the organoids or tissues are vascularized and are interconnected.

Accordingly, in certain embodiments, an in vitro system simulating mammalian organs comprising a microfluidic device, the microfluidic device comprising: (i) a first network comprising one or more channels connecting one or more chambers, and (ii) a second network comprising one or more channels connecting one or more chambers, or (c) a plurality of networks comprising one or more channels connecting one or more chambers, wherein each chamber comprises one or more organoids, cell populations, tissues or combinations thereof.

In certain embodiments, the microfluidic device further comprises a fluid inlet and outlet; a gas inlet and outlet; one or more connections to a device or operating system for measuring input and output values; one or more electrodes integrated within the microfluidic chip or combinations thereof. In a particular aspect, the fluid inlet and outlet will be a liquid inlet and outlet. In certain embodiments, the one or more organoids, cell populations, tissues or combinations thereof are contacted with a biological or chemical agent. In certain embodiments, the biological agent comprises growth factors, cytokines, enzymes, morphogens, antibodies, aptamers, drugs, hormones, peptides, proteins, oligonucleotides, polynucleotides, shRNA, siRNA, nanoparticles, mRNA, modified mRNA or combinations thereof. In certain embodiments, the chemical agent comprises small molecules, drugs, organic molecules, inorganic molecules, carbohydrates, synthetic compounds or combinations thereof. In certain embodiments, the channels optionally are interconnected to one or more other channels forming an interpenetrating vascular network or a branched interpenetrating vascular network. In certain embodiments, the organoid is created by culturing at least one of: pluripotent stem cells, multipotent stem cells, progenitor cells, terminally differentiated cells, endothelial cells, endothelial progenitor cells, immortalized cell lines, or primary cells. In certain embodiments, the organoid comprises: cerebral organoid, thyroid organoid, intestinal or gut organoid, hepatic organoid, pancreatic organoid, gastric organoid, kidney organoid, retinal organoid, cardiac organoid, bone organoid, thymus organoid, lymph node organoid, alveolar organoid or epithelial organoid. In certain embodiments, the organoid or tissue comprises a vascular network. In certain embodiments, the cell populations comprise pluripotent stem cells, induced pluripotent stem cells, multipotent stem cells, progenitor cells, terminally differentiated cells, endothelial cells, endothelial progenitor cells, immortalized cell lines, or primary cells.

In certain embodiments, microfluidic device comprises two or more chambers, wherein the two or more chambers are interconnected with one or more channels; a fluid inlet and outlet; a gas inlet and outlet; one or more connections to a device or operating system for measuring input and output values. In certain embodiments, the microfluidic device is a microfluidic chip. In certain embodiments, the microfluidic device further comprises one or more electrodes integrated within the microfluidic chip. In certain embodiments, cells, tissues organoids or combinations thereof, are cultured within each of the two or more chambers. In certain embodiments, the two or more chambers comprise different populations of cells, tissues organoids or combinations thereof. In certain embodiments, each of the two or more chambers are interconnected via one or more channels. In certain embodiments, each of the chambers and channels are sized to accommodate a desired organoid or populations of cells. In certain embodiments, the microfluidic device simulates any types of mammalian organs and vascular interconnections. In certain embodiments, the organoid or tissue comprises a vascular network.

In certain embodiments, a method of generating functional human or mammalian tissues or organoids, comprises culturing an organoid or a tissue construct wherein the microfluidic device comprises a first vascular network and a second vascular network, each vascular network comprising one or more interconnected vascular channels; exposing the organoid or a tissue construct to one or more biological agents, a biological agent gradient, a pressure, and/or an oxygen tension gradient, thereby inducing angiogenesis of capillary vessels to and/or from the tissue construct or organoid; and vascularizing the tissue construct or organoid, the capillary vessels connecting the first vascular network to the second vascular network, thereby creating a single vascular network and a perfusable tissue structure. The one or more biological agents include one or more of growth factors, morphogens, small molecules, drugs, hormones, DNA, shRNA, siRNA, nanoparticles, mRNA, and modified mRNA. The one or more interconnected vascular channels may be formed by a manufacturing process or by a biological developmental process that may include at least one of vasculogenesis, angiogenesis, or tubulogenesis. The one or more biological agents, the biological agent gradient, the pressure, and/or the oxygen tension gradient may further direct development, differentiation, and/or functioning of the tissue construct or organoid. In certain embodiments, the first vascular network and the second vascular network may be independently addressable. The first vascular network and the second vascular network may not be in contact with each other. In certain embodiments, the first vascular network and the second vascular network may be interconnected. In certain embodiments, a single vascular network may comprise an interpenetrating vascular network and/or a branched interpenetrating vascular network. The single vascular network may comprise interconnected arterial and venous channels. In certain embodiments, the tissue construct or organoid may be created by culturing at least one of: pluripotent stem cells, multipotent stem cells, progenitor cells, terminally differentiated cells, endothelial cells, endothelial progenitor cells, immortalized cell lines, or primary cells. In certain embodiments, the tissue construct or organoid may be created by culturing pluripotent or multipotent stem cells. The culturing may take place on a low-adhesion substrate, via a hanging drop method, via aggregation in microwells, via aggregation in microchannels, or by using a spinning bioreactor.

In the method, prior to, during and/or after culturing, the tissue construct or organoid may be further differentiated into a tissue containing at least one of pluripotent stem cells, multipotent stem cells, progenitor cells, terminally differentiated cells, endothelial cells, endothelial progenitor cells, immortalized cell lines, or primary cells. In certain embodiments, the t organoid may be a cerebral organoid, thyroid organoid, intestinal or gut organoid, hepatic organoid, pancreatic organoid, gastric organoid, kidney organoid, retinal organoid, cardiac organoid, bone organoid, cancer organoid, or epithelial organoid. In certain embodiments, the tissue construct or organoid may be exposed to the one or more biological agents and/or the biological agent gradient due to diffusion of the one or more biological agents within the tissue construct. Alternatively or in addition, the tissue construct or organoid may be exposed to the one or more biological agents and/or the biological agent gradient by localized deposition of materials loaded with the one or more biological agents within the microfluidic device. Alternatively or in addition, the tissue construct or organoid may be exposed to the one or more biological agents and/or the biological agent gradient by localized de-novo production of growth factors by localized protein translation. Alternatively or in addition, the tissue construct or organoid may be exposed to the one or more biological agents and/or the biological agent gradient via perfusion of one or both of the first and second vascular networks with the one or more biological agents. In certain embodiments, only one of the first and second vascular networks may be perfused with the one or more biological agents. In certain embodiments, both the first and second vascular networks may be perfused with the one or more biological agents, and a biological agent concentration in the first vascular network is different than a biological agent concentration in the second vascular network. In certain embodiments, both the first and second vascular networks are perfused with the one or more biological agents, and a biological agent concentration in the first vascular network is the same as a biological agent concentration in the second vascular network. The biological agents may include one or more of the following growth factors or small molecules: vascular endothelial growth factor (VEGF), basic fibroblast growth factor (bFGF), sphingosine-1-phosphate (S1P), phorbol myristate acetate (PMA), hepatocyte growth factor (HGF), monocyte chemotactic protein-1 (MCP-1), the angiopoietin ANG-1, the angiopoietin ANG-2, transforming growth factor beta (TGF-.beta.), epidermal growth factor (EGF), human growth factor, matrix metalloproteinases (MMP's), doxycycline, and histamine.

In certain embodiments, an oxygen partial pressure gradient may be introduced to one or both of the first and second vascular networks during perfusion. The oxygen partial pressure gradient may be formed by introducing deoxygenated media into one of the first and second vascular networks, and by introducing oxygenated media into the other of the first and second vascular networks. The media may be deoxygenated using either continuous bubbling of nitrogen gas through media, and/or by adding the enzymes glucose oxidase and catalase in the presence of glucose. The perfusion may be carried out at a flow rate of from about 1 microliter per minute to about 1 liter per minute. In certain embodiments, one or both of the first and second vascular networks may be subjected to a transmural pressure during the perfusion. In certain embodiments, the microfluidic device comprises a plurality of interconnected channels thereby providing a plurality of vascular networks. In certain embodiments, the tissue construct or organoid may be encapsulated in an extracellular matrix material. The extracellular matrix material may comprise a gel. In certain embodiments, the tissue construct or organoid may comprise a first population of cells or organoid cells and a second population of cells or organoid cells, where the cells or organoid may comprise at least two of: pluripotent stem cells, multipotent stem cells, progenitor cells, terminally differentiated cells, endothelial cells, endothelial progenitor cells, immortalized cell lines, neural cells, primary cells, or a combination thereof.

In certain embodiments, a method of identifying candidate therapeutic agents comprising contacting a microfluidic device or an in vitro system embodied herein with a candidate therapeutic agent and assaying for modulation of one or more biological parameters. In certain embodiments, the biological parameters comprise modulation of: gene expression, transcription, translation, receptor expression, biomarker expression, cell growth, cell death or combinations thereof.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity. The terminology used herein is for the purpose of describing particular embodiments only and is not intended to be limiting of the invention.

As used herein, the singular forms “a,” “an” and “the” are intended to include plural forms as well, unless the context clearly indicates otherwise. It will be further understood that the terms “comprises” or “comprising,” when used in this specification, specify the presence of stated features, steps, operations, elements components and/or groups or combinations thereof, but do not preclude the presence or addition of one or more other features, steps, operations, elements, components and/or groups or combinations thereof.

As used herein, the term “and/or” includes any and all possible combinations or one or more of the associated listed items, as well as the lack of combinations when interpreted in the alternative (“or”).

“Cells” as used herein are, in general, mammalian cells, such as dog, cat, cow, goat, horse, sheep, mouse, rabbit, rat, etc. cells. In some embodiments the cells are human cells. Suitable cells are known and are commercially available, and/or may be produced in accordance with known techniques. In some embodiments, the cells are harvested from a donor and passaged. In some embodiments, the cells are differentiated from cell lines. In some embodiments, the cells are derived from adult stem cells (bone marrow, peripheral blood, umbilical cord blood, Wharton's jelly in the umbilical cord or from placental tissues), embryonic stem cells, amniotic fluid stem cells, or any other source of stem cells that can be differentiated into the tissue of interest.

The term “embryoid body” refers to a plurality of cells containing pluripotent or multipotent stem cells formed into a three dimensional sphere, spheroid, or other three dimensional shape.

“Extracellular Matrix” (ECM) as used herein refers to extracellular molecules secreted by cells that provides structural and biochemical support to the surrounding cells. The ECM is normally composed of an interlocking mesh of fibrous proteins and polysaccharides such as glycosaminoglycans (GAGs). “Extracellular Matrix composition” as used herein refers to a composition including ECM proteins.

“Extracellular Matrix Proteins” (or “ECM proteins”) as used herein are known, and include but are not limited to those described in Y. Zhang et al., US Patent Application Publication No. 2013/0288375. Examples of ECM proteins include, but are not limited to, laminin, collagen type I, collagen type IV, fibronectin and elastin.

“Hydrogel” as used herein refers to naturally-derived hydrogels and synthetic hydrogels. Naturally-derived hydrogels and synthetic hydrogels may be mixed to form hybrid hydrogels. Naturally-derived hydrogels may include, but not limited to, Matrigel™, which is made out of native extracellular matrix proteins collected from a cell line, collagen and alginate. Naturally-derived hydrogels may be derived from decellularized tissue extracts. Extracellular matrix may be collected from a specific tissue and may be used as or combined with a hydrogel material to be used to support cells of that tissue type. See, e.g., Skardal et al., Tissue Specific Synthetic ECM Hydrogels for 3-D in vitro Maintenance of Hepatocyte Function, Biomaterials 33 (18): 4565-75 (2012). Chitosan hydrogel is an example of a naturally-derived hydrogel that is degradable and supportive for several different cell types. See, e.g., Moura et al., In Situ Forming Chitosan Hydrogels Prepared via Ionic/Covalent Co-Cross-Linking, Biomacromolecules 12 (9): 3275-84 (2011). Hyaluronic acid hydrogels may also be used. See, e.g., Skardal et al., A hydrogel bioink toolkit for mimicking native tissue biochemical and mechanical properties in bioprinted tissue constructs, Acta Biomater. 25:24-34 (2015). Synthetic hydrogels may be produced from a variety of materials (e.g., Poly-(ethylene glycol)) and using many techniques. In contrast to naturally-derived hydrogels, synthetic hydrogels may be produced uniformly and may be easily reproducible and characterized. Synthetic hydrogels may, however, lack some functional signals for cells, like the active sites found in natural extracellular matrix, limiting their potential to support cells. See, e.g., Mahoney et al., Three-Dimensional Growth and Function of Neural Tissue in Degradable Polyethylene Glycol Hydrogels, Biomaterials 27 (10): 2265-74 (2006). Hybrid hydrogels may offer a compromise and may allow for more control over the ability to reconstruct a specific microenvironment. By combining natural components, such as extracellular matrix molecules (e.g., extracellular matrix proteins), with defined synthetic hydrogels, a more easily reproducible and functional hydrogels can be produced. See, e.g., Salinas et al., Chondrogenic Differentiation Potential of Human Mesenchymal Stem Cells Photoencapsulated within Poly(Ethylene Glycol)—Arginine-Glycine-Aspartic Acid-Serine Thiol-Methacrylate Mixed-Mode Networks, Tissue Engineering 13 (5): 1025-34 (2007).

“Media” or “culture media” as used herein refers to an aqueous based solution that is provided for the growth, viability, or storage of cells used in carrying out the present invention. A media or culture media may be natural or artificial. A media or culture media may include a base media and may be supplemented with nutrients (e.g., salts, amino acids, vitamins, trace elements, antioxidants) to promote the desired cellular activity, such as cell viability, growth, proliferation, and/or differentiation of the cells cultured in the media. A “base media,” as used herein, refers to a basal salt nutrient or an aqueous solution of salts and other elements that provide cells with water and certain bulk inorganic ions essential for normal cell metabolism and maintains intra-cellular and/or extra-cellular osmotic balance. In some embodiments, a base media may include at least one carbohydrate as an energy source and/or a buffering system to maintain the medium within the physiological pH range. Examples of commercially available base media may include, but are not limited to, phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5A Medium, Medium 199, William's Medium E, insect media such as Grace's medium, Ham's Nutrient mixture F-10 (Ham's F-10), Ham's F-12, α-Minimal Essential Medium (αMEM), Glasgow's Minimal Essential Medium (G-MEM) and Iscove's Modified Dulbecco's Medium. See, e.g., US Patent Application Publication No. US20150175956.

“Mammalian” as used herein refers to both human subjects (and cells sources) and non-human subjects (and cell sources or types), such as dog, cat, mouse, monkey, etc. (e.g., for veterinary purposes).

“Organoid” as used herein refers to an artificial, in vitro construct created to mimic or resemble the functionality and/or histological structure of an organ or portion thereof. The cells used to produce embryoid bodies or organoids (including all further embodiments related thereto), are human cells or non-human primate cells, pluripotent stem cells, multipotent stem cells, progenitor cells, terminally differentiated cells, endothelial cells, endothelial progenitor cells, immortalized cell lines, or primary cells.

A “pluripotent” stem cell is not able to grow into an entire organism, but is capable of giving rise to cell types originating from all three germ layers, i.e., mesoderm, endoderm, and ectoderm, and may be capable of giving rise to all cell types of an organism. Pluripotency can be a feature of the cell per se, e.g. in certain stem cells, or it can be induced artificially. E.g. in certain embodiments, the pluripotent stem cell is derived from a somatic, multipotent, unipotent or progenitor cell, wherein pluripotency is induced, Such a cell is referred to as “induced pluripotent stem cell” or “iPSC” herein. The somatic, multipotent, unipotent or progenitor cell can, e.g., be used from a patient, which is turned into a pluripotent cell, that is subject to the described methods. Such a cell or the resulting tissue culture can be studied for abnormalities, e.g. during tissue culture development according to the described methods. A patient may, e.g., suffer from a neurological disorder or cerebral tissue deformity. Characteristics of the disorder or deformity can be reproduced in the described embryoid bodies or organoids and investigated. A “multipotent” cell is capable of giving rise to at least one cell type from each of two or more different organs or tissues of an organism, wherein the cell types may originate from the same or from different germ layers, but is not capable of giving rise to all cell types of an organism. In contrast, a “unipotent” cell is capable of differentiating to cells of only one cell lineage. A “progenitor cell” is a cell that, like a stem cell, has the ability to differentiate into a specific type of cell, with limited options to differentiate, with usually only one target cell. A progenitor cell is usually a unipotent cell, it may also be a multipotent cell, and often has a more limited proliferation capacity.

Additionally, the term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels (and some of these designs are shown by way of example, in the figures).

As used herein, the term “chamber” refers to a component in the microfluidic device that is enclosed to allow for the culture or cells or organoids and is connected via one or more channels either to one or more other chambers, or to a fluid inlet and outlet (e.g. a liquid inlet and outlet), or a gas inlet and outlet or combinations thereof. A chamber suitably may have a cross-sectional dimension greater than a microchannel, for example a cross-sectional dimension greater than 1 millimeter. In certain embodiments, a chamber suitably may have a cross-sectional greater than 1 millimeter.

As used herein, the term “channel” or “channels” are pathways (e.g. may be straight, curved, single, multiple, in a network, etc.) through a medium (e.g., silicon) that allow for movement of fluids such as liquids and gasses. Channels suitably can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid reservoir. Two components may be coupled to each other even though they are not in direct contact with each other. For example, two components may be coupled to each other through an intermediate component (e.g. tubing or other conduit).

Any compositions or methods provided herein can be combined with one or more of any of the other compositions and methods provided herein.

### BRIEF DESCRIPTION OF THE DRAWINGS

FIGS. 1A-1C are a series of photographs and a schematic showing an embodiment of a microfluidic chip. FIG. 1A shows a negative mold for the microfluidic chip.

FIG. 1B shows a stereolithography 3D printed negative mold made of PC-Like Advanced High Temp Translucent Amber (Accura 5530). FIG. 1C shows a completed PDMS based microfluidic chip following curing and bonding to a glass substrate demonstrating watertight channels.

FIGS. 2A-2E are a series of photographs, schematics and a cardiogram demonstrating that the microfluidic device is readily modifiable to suit a wide range of disease models and drug testing applications. FIG. 2A shows that electrodes can be integrated into the microfluidic chip so that excitable cells (e.g. cardiomyocytes, skeletal myocytes, or neural cells) can subsequently be paced, and functional outputs can be measured. FIG. 2B shows a brightfield image of adult cardiomyocytes seeded within chamber containing electrodes. FIG. 2C demonstrate the functional output data collected from cardiomyocytes being paced within their respective chamber. This system is modifiable as demonstrated by FIGS. 2D-2E. FIG. 2D shows a CAD design with larger channels allowing for larger numbers of organoids/cells to be seeded to maximize data output from single experiments FIG. 2E shows a CAD design containing three chambers to look at cross talk between three organ systems within one microfluidics chip.

FIGS. 3A-3D are a series of schematics and photographs demonstrating that organoids can readily be formed for use within the microfluidic device to achieve 3D cell culture FIG. 3A shows a workflow and timeline for generation of organoids. FIG. 3B shows purified differentiated iPSCs in 2D culture. FIG. 3C shows purified iPSCs spun down into wells to initiate organoid formation. FIG. 3D shows harvested iPSC derived organoids ready for use in a microfluidic chip.

FIGS. 4A-4B are series of photographs of various types of organoids demonstrating that organoids can be formed for use within the microfluidic device to evaluate multiple organ system interactions. FIG. 4A: Panel showing images of stains for DAPI and cTnT indicating organoids of a cardiac specific lineage. FIG. 4B: Panel showing images of stains for DAPI and A1AT indicating organoids of a hepatic specific lineage.

### DETAILED DESCRIPTION

New methods of creating embryoid bodies, organoids and tissue constructs suitable for studies of tissue development and disease, as well as transplantation are desired. Embryoid bodies or organoids (e.g., cerebral organoids) are a promising platform for studying tissue development processes (e.g., neurodevelopment processes) in a three dimensional, biomimetic environment.

A new approach is described in the present disclosure for creating tissue constructs, embryoid bodies or organoids. The present disclosure provides a system that can connect two different populations of cells (or group of cells-organoids) via a microcirculatory system. This design is similar to an organ-on-a-chip model with the addition of having a two or more organ (organoids) connected with a modeled circulatory system similar to the human body. Having organoids in circuit allows for determining the pathogenesis and testing therapeutic interventions on multiple organs. Current cell-based systems can test the effect a disease/therapy has on one cell type or a mixed cell type but not multiple organs in circulation, similar to human organs. The models embodied herein, have overcome this barrier to better recapitulate human physiology. This can be accomplished via microfluidic devices disclosed herein, including three-dimensional (3D) bioprinting.

### Microfluidic Devices

Microfluidic devices are described for high efficiency and throughput equipment having research, diagnostic and synthetic applications, among others. These devices may be used with real-time PCR equipment, fluorescent plate readers, robotic plate handlers, pipetting robots, and equipment designed to load, manipulate and read microfluidic devices, among other applications.

In certain aspects, the microfluidic devices may include an elastomeric layer and one or more rigid layers. One of the rigid layers may be a base layer that provides a thermal, electrical, physical, and/or optical interface between the device and surrounding equipment.

In certain aspects. the rigid layer opposite the base layer may be a translucent plastic layer that includes openings (e.g., wells) to accept samples and reagents delivered to the device. This layer may be made out of relatively inexpensive injection molded or thermoset plastic. The mold for this layer may also include recesses, channels and other structures that form part of fluid flow and mixing infrastructure of the device. For example, a surface of this layer that comes in contact with the elastomeric layer may include recesses that form part of mixing/reaction chambers, flow channels, and/or control channels in the microfluidic device.

In certain aspects, the elastomeric layer may be a single layer, or a plurality of layers bonded together. The elastomeric layer may include structure for all or part of the mixing/reaction chambers, flow channels, control channels, vent channels, deflectable membranes, check valves, and other components of the device.

In certain embodiments, the microfluidic device may comprise a hydrogel layer. In certain embodiments, the hydrogel layer is composed of a natural hydrogel material and/or a synthetic hydrogel material, the synthetic hydrogel material comprises one or more of polypropylene, polystyrene, polyacrylamide, polylactide, polyglycolide, polylactic acid, polylactic-co-glycolic acid, polyhydroxy acid, polylactic-co-glycolic acid, polydimethylsiloxane, polyanhydride, polyacid ester, polyamide, polyamino acid, polyacetal, polycyanoacrylate, polyurethane, polypyrrole, polyester, polymethacrylate, polyethylene, polycarbonate or polyethylene oxide, and the like, the natural hydrogel material comprises gelatin or derivatives thereof, alginate or derivatives thereof, cellulose or derivatives thereof, agar, matrigel, collagen or derivatives thereof, amino acid or derivatives thereof, glycoprotein and derivatives thereof, collagen or derivatives thereof, and the like, One or more of hyaluronic acid or a derivative thereof, chitosan or a derivative thereof, layer connecting protein, fibronectin, fibrin or a derivative thereof, silk fibroin or a derivative thereof, vitronectin, osteopontin, peptide fragment hydrogel and DNA hydrogel.

In certain embodiments, the porosity of the hydrogel material ensure good material exchange between cells and a culture environment, and are favorable for the long-term stable survival of the cells in the microfluidic device. The material provides attachment points for cells, and the cells can be adhered and migrated in the microstructure, so that the spatial arrangement and assembly of the cells are facilitated, and the organoid can be conveniently and better proliferated.

|00044| The footprint of the device and the arrangement of the mixing/reaction chambers may be compatible with an established format for automated laboratory equipment, such as the SBS format. Integrating the microfluidic devices with preexisting sample delivery and high efficiency and throughput testing equipment combines advantages from both fields. Microfluidic systems have fewer moving parts and simpler operational logistics than robotic fluid delivery systems. In general, the microfluidic systems cost less to manufacture and require less maintenance and repair. In addition, microfluidic systems can be manufactured with smaller sized conduits and chambers, allowing them to deliver smaller volumes of samples, reagents, etc., than practicable with, for example, pipetting robots. This can reduce the costs and waste products generated for large screening studies involving thousands or more combinations of reagents and samples. The small volumes can also make screening and combinatorial studies practical when only a small amount of a sample is available.

Smaller component dimensions also permit more densely packed arrangements of the reaction sites. For example, two, four, eight, or more microfluidic reaction chambers (each defining a reaction site) may be packed into the interrogation area of a single site for a standardized high throughput screening device. This can allow the microfluidic device to achieve a twofold, fourfold, eightfold. or more, increase in the throughput rate using an existing screening device.

A wide variety of methods and materials exists and will be known and appreciated by one of skill in the art for construction of microfluidic channels and networks thereof, such as those described, for example, in U.S. Pat. No. 8,047,829 and U.S. Patent Application Publication No. 20080014589, each of which is incorporated herein by reference in its entirety. For example, the microfluidic channel may be constructed using simple tubing, but may further involve sealing the surface of one slab comprising open channels to a second flat slab Materials into which microfluidic channels may be formed include silicon, glass, silicones such as polydimethylsiloxane (PDMS), and plastics such as poly(methyl-methacrylate) (known as PMMA or “acrylic”), cyclic olefin polymer (COP), and cyclic olefin copolymer (COC). The same materials can also be used for the second sealing slab. Compatible combinations of materials for the two slabs depend on the method employed to seal them together. The microfluidic channel may be encased as necessary in an optically clear material to allow for optical excitation (resulting in, e.g., fluorescence) or illumination (resulting in, e.g., selective absorption) of a sample as necessary, and to allow for optical detection of spectroscopic properties of light from a sample, as the sample is flowing through the microfluidic channel. Preferred examples of such optically clear materials that exhibit high optical clarity and low autofluorescence include, but are not limited to, borosilicate glass (e.g., SCHOTT BOROFLOAT® glass (Schott North America, Elmsford N.Y.)) and cyclo-olefin polymers (COP) (e.g., ZEONOR® (Zeon Chemicals LP, Louisville Ky.).

The microfluidic device can have a number of features. In one embodiment, said fluidic device further comprises at least one inlet port and at least one outlet port, and said culture media enters said inlet port and exits said outlet port. The combination of artificial construction and living materials allows modeling of physiological functions of tissues and organs.

Microfluidic culture systems are often made by ‘soft lithography’, a means of replicating patterns etched into silicon chips in more biocompatible and flexible materials. A liquid polymer, such as poly-dimethylsiloxane (PDMS), is poured on an etched silicon substrate and allowing it to polymerize into an optically clear, rubber-like material. This allows one to specify the shape, position and function of cells cultured on chips. Alternatively, inverting the PDMS mold and conformally sealing it to a flat smooth substrate, allows creation of open cavities, such as linear, hollow chambers, or ‘microfluidic channels’ for perfusion of fluids. Such PDMS culture systems are optically clear, allowing for high-resolution optical imaging of cellular responses. In some instances, miniaturized perfusion bioreactors for culturing cells are made by coating the surface of channels with extracellular matrix (ECM) molecules. Cells can be introduced via flow through the channel for capture and adherence to the ECM substrate. Additional details are found in Bhatia and Ingber, “Microfluidic organs-on-chips” Nat Biotechnol. (2014) 8:760-72, which is fully incorporated by reference herein.

Microfluidic chips provide control over system parameters in a manner not otherwise available in 3D static cultures or bioreactors. This allows study of a broad array of physiological phenomena. In some instances, integration of microsensors allows study of cultured cells in the microenvironmental conditions. Further, flow control of fluid in chips allows the generation of physical and chemical gradients, which can be exploited for study of cell migration, analysis of subcellular structure and cell-cell junctional integrity. In addition to detection and control of such mechanical forces, control of cell patterning allows study of physiological organization and interaction. For example, different cell types can be plated in distinct physical spaces, and using the above described techniques, shaped by micromolding techniques into organ-like forms, such as the villus shape of the intestine. Chips also allow the complex mechanical microenvironment of living tissues to be recapitulated in vitro. Cyclical mechanical strain can be introduced using flexible side chambers, with continuous rhythmic stretching relaxing lateral walls and attached central membranes. This cyclic mechanical deformation and fluid shear stresses introduced in parallel, mimic cellular exposure in living organs

### Organoids

In certain embodiments, the tissue constructs, embryoid bodies or organoids comprise a vasculature, multiple cell types and optionally other functional chemical substances, such as drugs, toxins, proteins and/or hormones, are programmably placed at desired locations within the device. This technique may lead to the rapid manufacturing of functional 3D tissues (i.e., “tissue constructs”) and organs needed for studies of tissue development and disease, as well as transplantation. The tissue constructs, embryoid bodies or organoids can also be used as a research tool to study the effects of any external (e.g. drugs or other stimuli) or internal (mutations) influences on growth and activity of cells in the tissue. Examples of organ, embryoid body, organoid, or tissue constructs that can be produced by the described methods include, but are not limited to, thyroid, pancreas, ureters, bladder, urethra, adrenal glands, lung, liver, pineal gland, pituitary gland, parathyroid glands, thymus gland, adrenal glands, appendix, gallbladder, spleen, prostate gland, reproductive organs, neural and vascular tissue.

As such, certain embodiments relate to methods of creating vascularized developing embryoid bodies or organoids (e.g., cerebral organoids) to enable nutrient delivery via perfusion necessary for generation of larger, more complex embryoid bodies or organoids for transplantation and drug screening applications, as well as for fundamental, long term studies of organogenesis.

In certain embodiments, the organoid is created by culturing initial populations of pluripotent or multipotent stem cells. In certain embodiments, the organoids can be obtained from culturing pluripotent stem cells. In principle, the cells may also be totipotent, if ethical reasons allow. A “totipotent” cell can differentiate into any cell type in the body, including the germ line following exposure to stimuli like that normally occurring in development. Accordingly, a totipotent cell may be defined as a cell being capable of growing, i.e. developing, into an entire organism.

The culturing methods are known in the art. For example, culturing can take place on a low-adhesion substrate (Doetschman T C, et al., The in vitro development of blastocyst-derived embryonic stem cell lines: formation of visceral yolk sac, blood islands and myocardium. J Embryol Exp Morphol 87:27-45 (1985)), via a hanging drop method (Reubinoff B E, et al., Embryonic stem cell lines from human blastocysts: somatic differentiation in vitro. Nat Biotechnol 18 (4): 399-404 (2002)), via aggregation in microwells (Mohr J C, et al., 3-D microwell culture of human embryonic stem cells. Biomaterials 27 (36): 6032-42 (2006), via aggregation in microchannels (Onoe H, et al., Differentiation Induction of Mouse Neural Stem Cells in Hydrogel Tubular Microenvironments with Controlled Tube Dimensions. Adv Healthc Mater. (2016), doi: 10.1002/adhm.201500903), or by using a spinning bioreactor (Carpenedo R L, et al., Rotary suspension culture enhances the efficiency, yield, and homogeneity of embryoid body differentiation. Stem Cells 25 (9): 2224-34 (2007)).

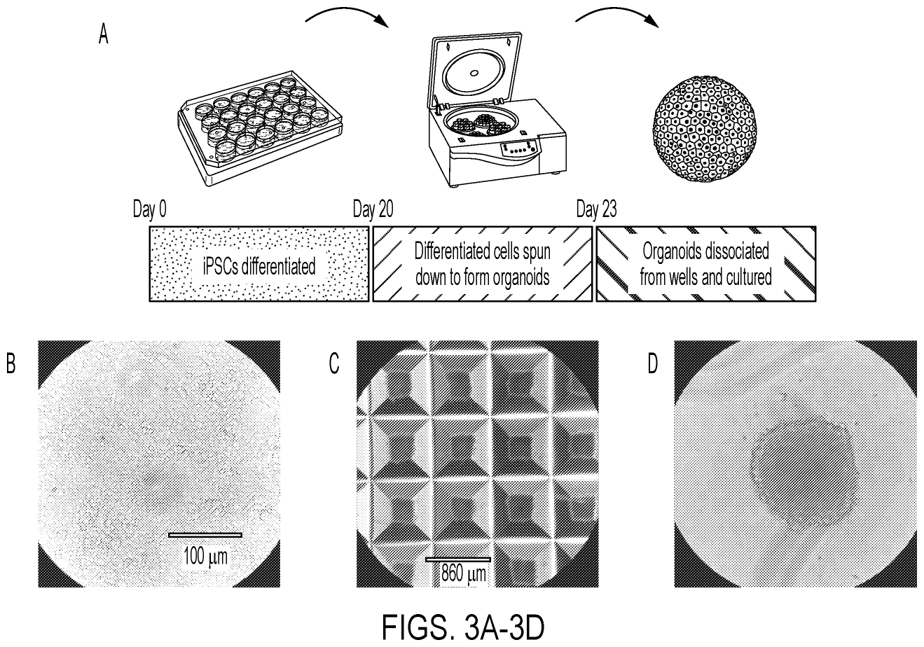

A typical organoid protocol, according to the described methods starts with isolated embryonic or pluripotent stem cells (e.g., induced pluripotent stem cells, or iPS cells, or iPSCs). FIG. 3A is a schematic representation showing a protocol that can be used in growing organoids. Organoids can readily be formed for use within the microfluidic devices embodied herein to achieve 3D cell culture. In certain embodiments, purified differentiated iPSCs are cultured in a 2D culture. Purified iPSCs are spun down into wells to initiate organoid formation. Harvested iPSC derived organoids are then ready for use in microfluidic chip. The organoid culture is in vitro grown (culturing step), i.e., it is not an isolated organ, such as brain or Kidney from an animal during any stages. Since it is grown from human pluripotent stem cells, this allows growth of human tissue without the need to obtain human fetal tissue samples.

For example, during the step of culturing the aggregate, the pluripotent stem cells can be induced to differentiate into a tissue (e.g., neural tissue) containing at least one of pluripotent stem cells, multipotent stem cells, progenitor cells, terminally differentiated cells, endothelial cells, endothelial progenitor cells, immortalized cell lines, or primary cells. For providing a multicellular aggregation, it is, e.g., possible to culture pluripotent stem cells from the multicellular aggregates. Exemplary media for culturing embryoid bodies or organoids include, but are not limited to, Aggrewell™ medium (AW) commercially available from StemCell Technologies, Inc., neural induction medium (NIM) comprising DMEM/F12 medium,

In certain embodiments, endothelial cells may be encouraged to undergo proliferation and specification to a brain microvascular phenotype by culturing the cells in brain microvascular endothelial cell (BMEC) medium.

In certain embodiments, various biological agents or factors may be used in combination with the media. Exemplary biological agents or factors that may be used in the described method include, e.g., basic FGF, noggin, the small molecule TGF-beta inhibitor SB431542, Activin A, BMP-4, Wnt, epidermal growth factor (EGF), ascorbic acid, retinoic acid, bovine brain extract, heparin, hydrocortisone, gentamicin, fetal bovine serum, Insulin-like growth factor (IGF), and vascular endothelial growth factor (VEGF).

In certain embodiments, relating to synthesizing, for example, cerebral organoids, during the development, the cell aggregates can form polarized neuroepithelial structures and a neuroepithelial sheet, which will develop several round clusters (rosettes). These steps can be controlled by neural induction medium as described by Eiraku (2008), US 2011/0091869 A1 and WO 2011/055855 A1.

In certain embodiments, standard differentiation media may be used. The method may include culturing in a three dimensional matrix, e.g. a gel, especially a rigid stable gel. As such, in certain embodiments, the method also includes a step of culturing the cell aggregates in a three dimensional matrix, such as a gel, which can result enhanced epithelial polarization and improved cortical layer formation. For example, further expansion of neuroepithelium and/or differentiation can be observed with embryoid bodies or organoids cultured in a three dimensional matrix.

A suitable three dimensional matrix may comprise collagen type 1 or matrigel. In certain embodiments, the three dimensional matrix comprises extracellular matrix from the Engelbreth-Holm-Swarm tumor or any component thereof such as laminin, collagen, preferably type 4 collagen, entactin, and optionally further heparan-sulfated proteoglycan or any combination thereof. Such a matrix is Matrigel. Matrigel was previously described in U.S. Pat. No. 4,829,000, which is incorporated by reference in its entirety.

In certain embodiments, the three dimensional matrix may be a three dimensional structure of a biocompatible matrix. It may include collagen, gelatin, chitosan, hyaluronan, methylcellulose, laminin and/or alginate. The matrix may be a gel, in particular a hydrogel. Organo-chemical hydrogels may comprise polyvinyl alcohol, sodium polyacrylate, acrylate polymers and copolymers with an abundance of hydrophilic groups. Hydrogels comprise a network of polymer chains that are hydrophilic, sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. After the expansion, the cell aggregates can be cultured in suspension culture, such as a bioreactor.

The organoids/cells can be seeded onto the microfluidic devices. The microfluidic devices are readily modifiable to suit a wide range of disease models and drug testing applications. For example, electrodes can be integrated into the microfluidic chip so that excitable cells (e.g. cardiomyocytes, skeletal myocytes, or neural cells) can subsequently be paced, and functional outputs can be measured (FIG. 2A). Prior to, during and/or after the depositing or embedding, the embryoid body or organoid is further differentiated using a combination of NIM, NDM1, EGM-2 or NDM2 media into a tissue containing at least one of pluripotent stem cells, multipotent stem cells, progenitor cells, terminally differentiated cells, endothelial cells, endothelial progenitor cells, immortalized cell lines, or primary cells.

In certain embodiments, stem cells (e.g., iPSCs) are cultured to form a cell aggregate, the pluripotent stem cells can be induced to differentiate into a tissue (e.g., neural tissue) containing at least one of pluripotent stem cells, multipotent stem cells, progenitor cells, terminally differentiated cells, endothelial cells, endothelial progenitor cells, immortalized cell lines, or primary cells. For providing a multicellular aggregation, it is, e.g., possible to culture pluripotent stem cells from the multicellular aggregates.

In certain embodiments, organoids comprising at least two different populations of organoid can be produced and later vascularized. For example, in certain embodiments, the organoid can comprise multiple populations of cells, e.g., at least two different cell lineages, such as endothelial and neuronal, obtained by differentiation of iPSCs using the same culture condition.

In certain embodiments, the method of producing the multi-population organoids includes culturing a wild-type population of cells and a genetically-engineered inducible population of cells in a medium, inducing direct differentiation and/or transdifferentiation of the genetically-engineered inducible population of cells into the first population of the organoid cells, inducing differentiation of the wild-type population of cells into the second population of the organoid cells, and thereby forming the e organoid comprising at least the first population of the organoid cells and the second population of the organoid cells.

The terms “direct differentiation” or “directed differentiation” refer to the culture of pluripotent or multipotent stem cells in a condition that preferentially encourages the differentiation of the stem cell to a specific, more differentiated state. For example, a pluripotent stem cell may be cultured in a condition that results in an enriched population of specific multipotent stem cells such as neural progenitor cells. Alternatively, a multipotent stem cell such as a neural stem cell may be directly differentiated into a more differentiated state such as a neuron, astrocyte or oligodendrocyte. The term “transdifferentiation” refers to the conversion of one cell type that may be a multipotent or unipotent stem cell, or a terminally differentiated mature cell phenotype to a different cell type that may be a different multipotent or unipotent stem cell, or a terminally differentiated mature cell phenotype. For example, a neural stem cell, a radial glia, or a neuron may be transdifferentiated into an endothelial cell.

The genetically-engineered inducible population of cells may be created by introducing a DNA delivery element comprising at least one of constitutive promoter, small molecule inducible promoter, cell-autonomous promoter, cell non-autonomous promoter, selection marker, or a combination thereof. Examples of constitutive promoters include, e.g., EF1alpha, PGK, Ubiquitin, and CMV. Examples of small molecule inducible promoters include, e.g. doxycycline or cumate inducible promoters. Examples of cell-autonomous promoters include, e.g., cell type-specific promoters, such as DCX. Examples of cell non-autonomous promoter include, e.g., heat induced and light induced promoters. DNA delivery elements can be selected from lentiviral inverted repeats, packaging signal (e.g., pLIX403 vector), transposon integration elements (e.g., PiggyBac vector), episomal replication elements. Alternatively, transient expression by electroporation or lipofection can be used. Selection markers may be selected from, e.g., drug resistance markers (e.g. puromycin, neomycin, and blasticidin). Alternatively, transient expression followed by dilution from cell division rather than selection markers may be used.

Examples of specific transcription factors that may be used to induce endothelial cells within any organoid (e.g. for vasculature) and to produce mixed populations within organoids include ETV2/ER71, FL11, ERG, which induce differentiation of mature amniotic cells to endothelial cells; Gata2, FOXC1, FOXC2, HEY1, HEY2, SOX7, SOX18, PROX1, which induce differentiation of stem cells into various subtypes of endothelial cells (e.g. venous, arterial, lymphatic); Brachyury/T, which may be used for possible mesoderm induction, required for primitive streak formation in vivo.

Examples of specific transcription factors that may be used to induce neurons within any organoid (e.g. autonomic nervous system control of internal organs) include NEUROG1/2 (Busskamp, et al. “Rapid neurogenesis through transcriptional activation in human stem cells.” Molecular systems biology10.11 (2014): 760.), which induce formation of excitatory neurons; ASCL1 (Chanda, et al. “Generation of induced neuronal cells by the single reprogramming factor ASCL1.” Stem cell reports 3.2 (2014): 282-296.), which induce formation of excitatory neurons; ASCL1, BRN2, MYT1L, LHX3, HB9, ISL1, NGN2 (Son, Esther Y., et al. “Conversion of mouse and human fibroblasts into functional spinal motor neurons.” Cell stem cell 9.3 (2011): 205-218.), which induce formation of motor neurons; and ASCL1, MYT1L, KLF7 (Wainger, Brian J., et al. “Modeling pain in vitro using nociceptor neurons reprogrammed from fibroblasts.” Nature neuroscience 18.1 (2015): 17-24.), which induce formation of pain receptor neurons.

Accordingly, in certain embodiments, the first population of the organoid cells can comprise pluripotent stem cells, multipotent stem cells, progenitor cells, terminally differentiated cells, endothelial cells, endothelial progenitor cells, immortalized cell lines, or primary cells. The second population of the organoid cells comprises neural progenitor cells. The neural progenitor cells can form at least one of excitatory neurons, inhibitory interneurons, motor neurons, dopaminergic neurons, pain receptor neurons, astrocytes, oligodendrocyte progenitor cells, oligodendrocytes.

The step of inducing direct differentiation and/or transdifferentiation of the genetically-engineered inducible population of cells can comprise introducing at least one cue selected from the group consisting of transcription factors, drugs, small molecules, growth factors, morphogens, hormones, DNA, shRNA, siRNA, nanoparticles, mRNA, modified mRNA, heat, light, and mechanical stimulation. In certain embodiments, the direct differentiation may be accompanied by a secondary induction of a different gene, e.g., a second orthogonal induction. This secondary induction may occur at an earlier time, simultaneously, or at a later time than the first gene induction. The secondary gene induction may be via providing at least one cue selected from the group consisting of transcription factors, drugs, small molecules, growth factors, morphogens, hormones, DNA, shRNA, siRNA, nanoparticles, mRNA, modified mRNA, heat, light, and mechanical stimulation.

In certain embodiments, the cue selected for the secondary gene induction is the same as the cue selected for the step of inducing direct differentiation and/or transdifferentiation of the genetically-engineered inducible population of cells. Alternatively, the cue selected for the secondary gene induction is different, and orthogonal, from the cue selected for the step of inducing direct differentiation and/or transdifferentiation of the genetically-engineered inducible population of cells. The step of culturing may be in a differentiation medium. In certain embodiments, the differentiation medium includes one or more agents.

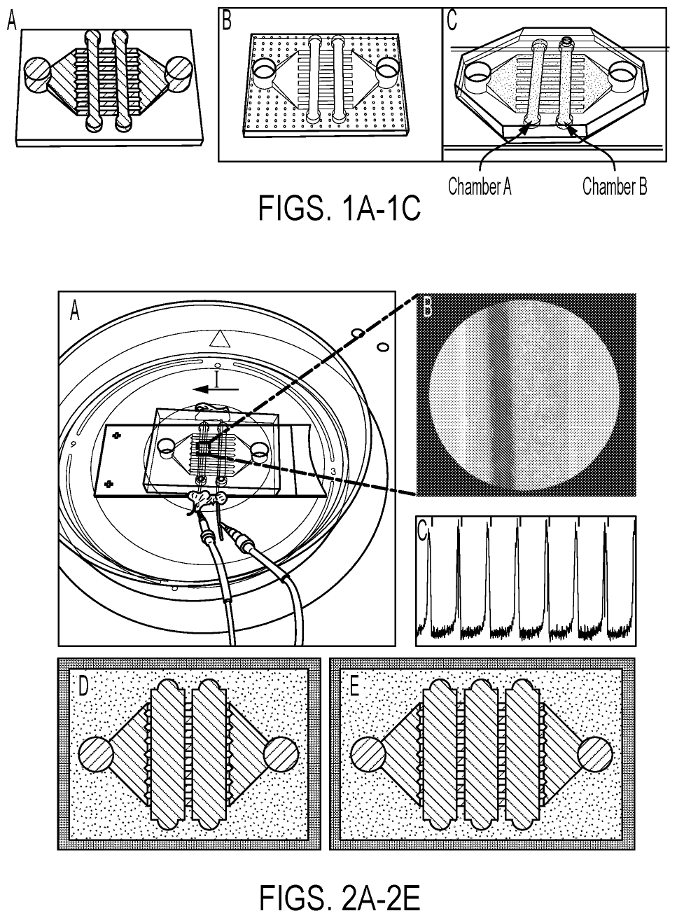

Once, or before, the organoids grows to a size at which it becomes oxygen or nutrient limited, the organoid is implanted or embedded onto a microfluidic device (FIGS. 2A-2E and 3A). The microfluidic device can comprise interconnecting channels which can mimic a vascular system. In general, a microfluidic device may include one or more chambers each dimensioned to accept the organoids such that a first chamber is in fluid contact with a second chamber and the like. The fluid may be a liquid such as media, or a gas such as air. The device may further include a fluid inlet and fluid outlet for each chamber, fluid reservoirs connected therewith, etc. In some embodiments, a microfluidic device may be provided in the form of a cartridge for “plug in” or insertion into a larger apparatus including pumps, culture media reservoir(s), detectors, and the like. FIGS. 1A to 1C show microfluidic chips which were generated using a computer aided design (CAD) file of a negative mold which is subsequently 3D printed and used to generate polydimethylsiloxane (PDMS) based chips. The substrates onto which the cells or organoids are deposited can comprises a material such as glass or other ceramics, PDMS, acrylic, polyurethane, polystyrene or other polymers. In some embodiments, the substrate may comprise living tissue or dehydrated tissue, or one of the extracellular matrix compositions described above. The substrate may be cleaned and surface treated prior to printing. For example, glass substrates may undergo a silane treatment to promote bonding of the cell-laden filaments to the glass substrate. In some embodiments, it is envisioned that the substrate may not be a solid-phase material but may instead be in the liquid or gel phase and may have carefully controlled rheological properties, as described, for example, in W. Wu et al., Adv. Mater. 23 (2011) H178-H183.

In certain embodiments, the organoid may be encapsulated in an extracellular matrix material. The extracellular matrix material may comprise a gel. Additional examples of matrices that may be used for encapsulating the embryoid body or organoid include, but are not limited to, at least one of collagen I, fibrin, matrigel, gelatin, gelatin methacrylate, laminin, carbopol, NIPAM, PEG, PHEMA, silk, hyaluronic acid, or combinations thereof.

In certain embodiments, the organoid is then exposed to one or more biological agents or factors, a biological agent gradient, a pressure, and/or an oxygen tension gradient, thereby inducing angiogenesis of capillary vessels to and/or from the embryoid body or organoid

In certain embodiments, growth factors and oxygen may be directly supplied to grow embryoid bodies or organoids via perfusion. Some examples of growth factors that encourage connection of vasculature include, but are not limited to, vascular endothelial growth factor (VEGF), basic fibroblast growth factor (bFGF), sphingosine-1-phosphate (S1P), phorbol myristate acetate (PMA), hepatocyte growth factor (HGF), monocyte chemotactic protein-1 (MCP-1), the angiopoietin ANG-1, the angiopoietin ANG-2, transforming growth factor beta (TGF-. beta.), epidermal growth factor (EGF), human growth factor, matrix metalloproteinases (MMP's), and histamine.

In certain embodiments, the organoid is exposed to the one or more biological agents and/or the biological agent gradient due to diffusion of the one or more biological agents. Alternatively, the organoid is exposed to the one or more biological agents and/or the biological agent gradient by localized deposition of materials. Alternatively, the organoid is exposed to the one or more biological agents and/or the biological agent gradient by localized de-novo production of growth factors by localized protein translation. Alternatively, the organoid is exposed to the one or more biological agents and/or the biological agent gradient via perfusion.

The organoids as described herein may be used as an alternative to live animal testing for compound or vaccine screening (e.g., screening for efficacy, toxicity, or other metabolic or physiological activity) or for treatment of (including resistance to treatment of) infection or disease. For acute treatment testing, compound or vaccine may be applied, e.g., once for several hours. For chronic treatment testing, compound or vaccine may be applied, e.g., for days to one week. Such testing may be carried out by providing an organoid as described herein under conditions which maintain constituent cells of that organoid alive (e.g., in a culture media with oxygenation); applying a compound to be tested (e.g., a drug candidate) to the organoid (e.g., by topical or vapor application to the epithelial layer); and then detecting a physiological response (e.g., damage, scar tissue formation, infection, cell proliferation, burn, cell death, marker release such as histamine release, cytokine release, changes in gene expression, etc.), the presence of such a physiological response indicating said compound or vaccine has therapeutic efficacy, toxicity, or other metabolic or physiological activity if inhaled or otherwise delivered into the lung of a mammalian subject. A control sample of the organoid may be maintained under like conditions, to which a control compound (e.g., physiological saline, compound vehicle or carrier) may be applied, so that a comparative result is achieved, or damage can be determined based on comparison to historic data, or comparison to data obtained by application of dilute levels of the test compound, etc.

It will be understood that the organoids as described herein can be an excellent tool to study drug delivery since the organoids can include both an epithelial cell layer and an endothelial cell layer. The endothelial cell layer of the organoids may be exposed to a liquid (e.g., media) and may function as a mature vascular barrier that controls materials passing through the endothelial cell layer. The epithelial cell layer of the organoids may be exposed to a gas (e.g., air) or liquid and thus may be exposed to materials delivered by aerosol.

Methods of determining whether a test compound has immunological activity may include testing for immunoglobulin generation, chemokine generation and cytokine generation by the cells.

Once produced, the devices described above may be used immediately, or prepared for storage and/or transport. To store and transport the device, a transient protective support media that is a flowable liquid at room temperature (e.g., 25° C.), or gels or solidifies at refrigerated temperatures (e.g., 4° C.), such as a gelatin mixed with water, may be added into the device to substantially or completely fill the chamber(s), and preferably also any associated conduits. Any inlet and outlet ports may be capped with a suitable capping element (e.g., a plug) or capping material (e.g., wax). The device may be then packaged together with a cooling element (e.g., ice, dry ice, a thermoelectric chiller, etc.) and all may be placed in a (preferably insulated) package.

In some embodiments, to store and transport the device, a transient protective support media that is a flowable liquid at cooled temperature (e.g., 4° C.), but gels or solidifies at warm temperatures such as room temperature (e.g., 20° C.) or body temperature (e.g., 37° C.), such as poly(N-isopropylacrylamide) and poly(ethylene glycol) block co-polymers, may be added into the device to substantially or completely fill the chamber(s), and preferably also any associated conduits.

Upon receipt, the end user may simply remove the device from the associated package and cooling element, may allow the temperature to rise or fall (depending on the choice of transient protective support media), may uncap any ports, and may remove the transient protective support media with a syringe (e.g., by flushing with growth media).

### Examples

Microfluidic chips were designed by generating a computer aided design (CAD) file of a negative mold which is subsequently 3D printed and used to generate polydimethylsiloxane (PDMS) based chips. A stereolithography 3D printed negative mold made of PC-Like Advanced High Temp Translucent Amber (Accura 5530) was prepared following curing and bonding to a glass substrate demonstrating watertight channels (FIGS. 1A-1C).

The microfluidic devices were readily modifiable to suit a wide range of disease models and drug testing applications (FIGS. 2A, 2D 2E). For example, electrodes can be integrated into the microfluidic chip so that excitable cells (e.g. cardiomyocytes, skeletal myocytes, or neural cells) can subsequently be paced, and functional outputs can be measured (FIG. 2A). The microfluidic device can be manufactured with larger channels allowing for larger numbers of organoids/cells to be seeded to maximize data output from single experiments (FIG. 2D). The microchip can be designed to contain three chambers to look at cross talk between three organ systems within one microfluidics chip (FIG. 2E).

FIG. 2B shows a brightfield image of adult cardiomyocytes seeded within chamber containing electrodes. Functional output data collected from cardiomyocytes being paced within their respective chamber (FIG. 2C).

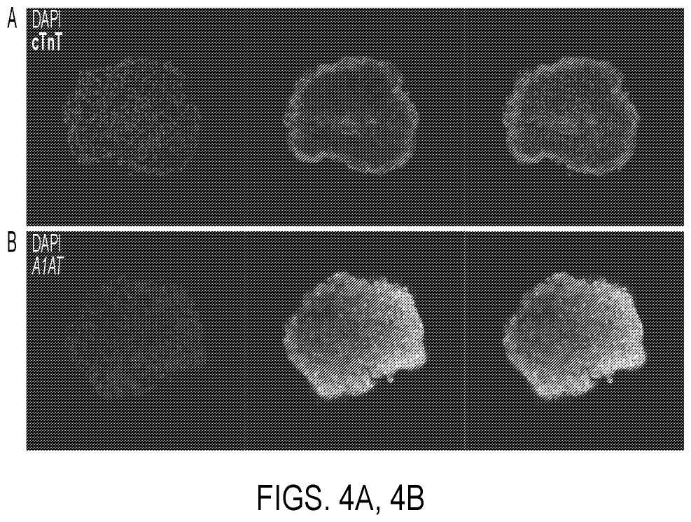

Organoids can readily be formed for use within the microfluidic device to achieve 3D cell culture as shown in FIGS. 3A-3D. Various types of organoids can be formed for use within the microfluidic device to evaluate multiple organ system interactions (FIGS. 4A, 4B).

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. An in vitro system simulating mammalian organs comprising a microfluidic device, the microfluidic device comprising: (i) a first network comprising one or more channels connecting one or more chambers, and (ii) a second network comprising one or more channels connecting one or more chambers, or (c) a plurality of networks comprising one or more channels connecting one or more chambers, wherein each chamber comprises one or more organoids, cell populations, tissues or combinations thereof.

2. An in vitro system simulating mammalian organs comprising a microfluidic device, the microfluidic device comprising:
(i) a first network comprising one or more channels connecting one or more chambers, and (ii) a second network comprising one or more channels connecting one or more chambers,
wherein each chamber comprises one or more organoids, cell populations, tissues or combinations thereof.

3. The in vitro system of claim 1, wherein the microfluidic device further comprises a fluid inlet and outlet; a gas inlet and outlet; one or more connections to a device or operating system for measuring input and output values; one or more electrodes integrated within the microfluidic chip or combinations thereof.

4. The in vitro system of claim 1, wherein the one or more organoids, cell populations, tissues or combinations thereof are contacted with a biological or chemical agent.

5. The in vitro system of claim 4, wherein the biological agent comprises growth factors, cytokines, enzymes, morphogens, antibodies, aptamers, drugs, hormones, peptides, proteins, oligonucleotides, polynucleotides, shRNA, siRNA, nanoparticles, mRNA, modified mRNA or combinations thereof.

6. The in vitro system of claim 4 or 5, wherein the chemical agent comprises small molecules, drugs, organic molecules, inorganic molecules, carbohydrates, synthetic compounds or combinations thereof.

7. The in vitro system of claim 1, wherein the channels optionally are interconnected to one or more other channels forming an interpenetrating vascular network or a branched interpenetrating vascular network.

8. The in vitro system of claim 1, wherein the organoid is created by culturing at least one of: pluripotent stem cells, multipotent stem cells, progenitor cells, terminally differentiated cells, endothelial cells, endothelial progenitor cells, immortalized cell lines, or primary cells.

9. The in vitro system of claim 1, wherein the organoid comprises: cerebral organoid, thyroid organoid, intestinal or gut organoid, hepatic organoid, pancreatic organoid, gastric organoid, kidney organoid, retinal organoid, cardiac organoid, bone organoid, thymus organoid, lymph node organoid, alveolar organoid or epithelial organoid.

10. The in vitro system of claim 1, wherein the organoid or tissue comprises a vascular network.

11. The in vitro system of claim 1, wherein the cell populations comprise pluripotent stem cells, induced pluripotent stem cells, multipotent stem cells, progenitor cells, terminally differentiated cells, endothelial cells, endothelial progenitor cells, immortalized cell lines, or primary cells.

12. A microfluidic device comprising:
two or more chambers, wherein the two or more chambers are interconnected with one or more channels;
a fluid inlet and outlet;
a gas inlet and outlet;
one or more connections to a device or operating system for measuring input and output values.

13. The microfluidic device of claim 12, wherein the microfluidic device is a microfluidic chip.

14. The microfluidic device of claim 12, further comprising one or more electrodes integrated within the microfluidic chip.

15. The microfluidic device of claim 12, wherein cells, tissues organoids or combinations thereof, are cultured within each of the two or more chambers.

16. The microfluidic device of claim 15, wherein the two or more chambers comprise different populations of cells, tissues organoids or combinations thereof.

17. The microfluidic device of claim 15, wherein each of the two or more chambers are interconnected via one or more channels.

18. The microfluidic device of claim 12, wherein each of the chambers and channels are sized to accommodate a desired organoid or populations of cells.

19. The microfluidic device of claim 12, wherein the microfluidic device simulates any types of mammalian organs.

20. (canceled)

21. A method of identifying candidate therapeutic agents comprising contacting the microfluidic device of claim 1 with a candidate therapeutic agent and assaying for modulation of one or more biological parameters.

22. (canceled)
